(19) United States
(12) Patent Application Publication
(10) Pub. No.: US 2022/0023476 A1
(43) Pub. Date: Jan. 27, 2022

(54) Removal of Pathogens in an Environment

(71) Applicant: Paerosol, Mt. Pleasant, SC (US)

(72) Inventors: Steven Gareleck, Mt. Pleasant, SC (US); Brian Domrese, Mt. Pleasant, SC (US); Frederick Scott Alderson, Mt. Pleasant, SC (US)

(73) Assignee: Paerosol, Mt. Pleasant, SC (US)

(21) Appl. No.: 16/938,357

(22) Filed: Jul. 24, 2020

(51) Int. Cl. A61L 2/22

(52) U.S. Cl. CPC A61L 2/22; A61L 2101/20

(57) ABSTRACT

A method for providing a reduced pathogen environment is provided. The method includes providing a decontamination fluid and micro-aerosolizing the decontamination fluid within the environment for a predetermined period of time based on a volume of air in the environment, wherein the micro-aerosolized decontamination fluid has a particle size of between about 0.2 and about 1 micron.

### BACKGROUND

The present invention generally relates to pathogen removal and more specifically, to removal of pathogens in an environment.

As coronavirus, and other pathogens, spread in the environment, removal of these pathogens has become a top priority. Various methods and devices have been used to attempt to destroy pathogens, but each of the currently available methods, such as spraying disinfectant or utilizing ultraviolet light, fails to reach the majority of pathogens that are not disposed on top surfaces in the environment. The presence of these less accessible pathogens creates a hazard for anyone present in the environment.

### SUMMARY

Embodiments of the present invention are directed to a method for providing a reduced pathogen environment. The method includes providing a decontamination fluid and micro-aerosolizing the decontamination fluid within the environment for a predetermined period of time based on a volume of air in the environment, wherein the micro-aerosolized decontamination fluid has a particle size of between about 0.2 and about 1 micron.

Further embodiments of the present invention are directed to micro-aerosol generator (“MAG”) including a vessel body and a nozzle assembly mounted within the vessel body for micro-aerosolizing a decontamination fluid using compressed air. The MAG also includes a first particle adjustment trap on the vessel body for reducing a size of particles micro-aerosolized by the nozzle assembly and emitted by the MAG.

Further embodiments of the invention are directed to a MAG having a vessel body and a nozzle assembly mounted within the vessel body for micro-aerosolizing a decontamination fluid using compressed air. The nozzle assembly includes a nozzle base, a nozzle o-ring on the nozzle base, a nozzle top cover on the nozzle o-ring, a variable adjustment tip having a decontamination fluid inlet for receiving decontamination fluid and an outlet for releasing decontamination fluid, and an adjustable stand-off for adjusting a height of the variable adjustment tip.

Additional technical features and benefits are realized through the techniques of the present invention. Embodiments and aspects of the invention are described in detail herein and are considered a part of the claimed subject matter. For a better understanding, refer to the detailed description and to the drawings.

### BRIEF DESCRIPTION OF THE DRAWINGS

The specifics of the exclusive rights described herein are particularly pointed out and distinctly claimed in the claims at the conclusion of the specification. The foregoing and other features and advantages of the embodiments of the invention are apparent from the following detailed description taken in conjunction with the accompanying drawings in which:

FIG. 1 depicts a micro-aerosol generator according to an embodiment of the present invention;

FIG. 2 depicts a particle adjustment trap that is a part of a bowl assembly of the micro-aerosol generator according to an embodiment of the present invention; and

FIG. 3 depicts a variable adjustment tip according to an embodiment of the present invention.

The diagrams depicted herein are illustrative. There can be many variations to the diagrams, or the operations described therein, without departing from the spirit of the invention. For instance, the actions can be performed in a differing order or actions can be added, deleted or modified. Also, the term “coupled,” and variations thereof, describe having a communications path between two elements and does not imply a direct connection between the elements with no intervening elements/connections between them. All of these variations are considered a part of the specification.

In the accompanying figures and following detailed description of the disclosed embodiments, the various elements illustrated in the figures are provided with two or three digit reference numbers. With minor exceptions, the leftmost digit(s) of each reference number correspond to the figure in which its element is first illustrated.

### DETAILED DESCRIPTION

Various embodiments of the invention are described herein with reference to the related drawings. Alternative embodiments of the invention can be devised without departing from the scope of this invention. Various connections and positional relationships (e.g., over, below, adjacent, etc.) are set forth between elements in the following description and in the drawings. These connections and/or positional relationships, unless specified otherwise, can be direct or indirect, and the present invention is not intended to be limiting in this respect. Accordingly, a coupling of entities can refer to either a direct or an indirect coupling, and a positional relationship between entities can be a direct or indirect positional relationship. Moreover, the various tasks and process steps described herein can be incorporated into a more comprehensive procedure or process having additional steps or functionality not described in detail herein.

The following definitions and abbreviations are to be used for the interpretation of the claims and the specification. As used herein, the terms “comprises,” “comprising,” “includes,” “including,” “has,” “having,” “contains” or “containing,” or any other variation thereof, are intended to cover a non-exclusive inclusion. For example, a composition, a mixture, process, method, article, or apparatus that comprises a list of elements is not necessarily limited to only those elements but can include other elements not expressly listed or inherent to such composition, mixture, process, method, article, or apparatus.

Additionally, the term “exemplary” is used herein to mean “serving as an example, instance or illustration.” Any embodiment or design described herein as “exemplary” is not necessarily to be construed as preferred or advantageous over other embodiments or designs. The terms “at least one” and “one or more” may be understood to include any integer number greater than or equal to one, i.e. one, two, three, four, etc. The terms “a plurality” may be understood to include any integer number greater than or equal to two, i.e. two, three, four, five, etc. The term “connection” may include both an indirect “connection” and a direct “connection.”

The terms “about,” “substantially,” “approximately,” and variations thereof, are intended to include the degree of error associated with measurement of the particular quantity based upon the equipment available at the time of filing the application. For example, “about” can include a range of ±8% or 5%, or 2% of a given value.

For the sake of brevity, conventional techniques related to making and using aspects of the invention may or may not be described in detail herein. In particular, various aspects of computing systems and specific computer programs to implement the various technical features described herein are well known. Accordingly, in the interest of brevity, many conventional implementation details are only mentioned briefly herein or are omitted entirely without providing the well-known system and/or process details.

Embodiments of the present invention use a micro-aerosol generator (“MAG”) to disperse a micro-aerosol, a gas-like fog, with particle size ranging from 0.1 microns to 2 microns in diameter. Micro-aerosols provide advantages over traditional foggers in that foggers typically have a particle size of from five to 50 microns, with an average size of 20 microns. Thus, a micro-aerosol is approximately twenty times smaller on average than a fog.

Since the micro-aerosol particles are small, they are widely dispersed using diffusion going from areas of high concentration to low concentration. The particles act like a gas and evenly distribute. It is unnecessary to “spray” or “fog” and labor is significantly reduced compared to traditional foggers and sprayers. As micro-aerosol particles average one micron, they clean both air and surface without getting surfaces wet or affecting electronics. When particles are less than 0.7 microns in diameter, they go through soft materials, such as clothing, drapes, and carpets. As they pass through fabrics and into carpeting, they are disinfecting where traditional fog particles have no access. The micro-aerosol particles kill odors, since they can get to bacteria, mold, and viruses present throughout the environment.

The MAG consistent with embodiments of the present invention disperses a micro-aerosol in ranges from 0.1 to 2 microns, with an average particle size of 1 micron. Because of the particle size, the particles follow Graham's Law going from areas of high concentration to areas of low concentration diffusing through the environment and covering virtually all areas. Using a particle adjustment trap (“PAT”) placed as an exterior portion of the MAG, the size of the particles can be fine-tuned. Additionally, a variable adjustment tip (“VAT”) placed within a nozzle assembly of the MAG may also be used to adjust particle size. While either feature alone may be present in order to adjust particle size, using the PAT and VAT in the same MAG allows for the widest range of adjustment.

In exemplary embodiments consistent with the present invention, Paeroltye, a proprietary from of hypochlorous acid (“HOCL”) is used as the micro-aerosol. Paerolyte is an all-natural organic disinfectant that is eighty times stronger than bleach. Paerolyte is PH neutral, non-toxic to humans, and safe on skin, but it kills up to 99.9999% of mold, bacteria, and viruses with which it comes in contact. While Paerolyte is used in an exemplary embodiment of the invention, those skilled in the art having read this disclosure will appreciate that other forms of HOCL and other substances can be used with the MAG described herein. However, HOCL has particular benefits due to its use of phagocytosis that uses HOCL's neutral electrical charge to penetrate and destroy negatively charged cell walls of bacteria and viruses. The free flowing particles of HOCL are attracted to the pathogens like a magnet, quickly attacking them where they then oxidize and are destroyed. Only particles in this size range can act like a gas and cover virtually all areas in an enclosed space.

The MAG used in combination with HOCL produces a reduced pathogen environment within confined spaces. Thus, it is suitable for use in classrooms, hospitals, airplanes, offices, and any space needing cleansing.

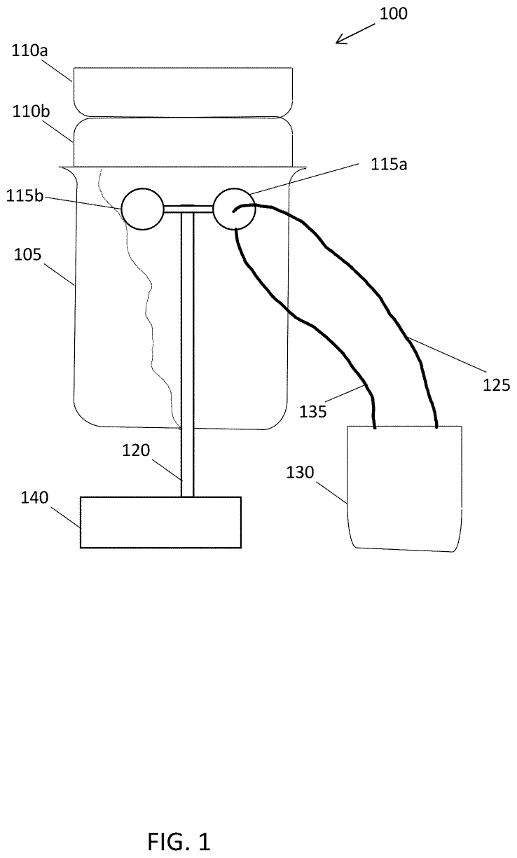

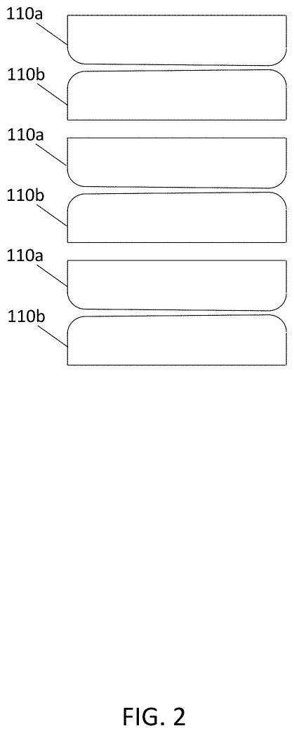

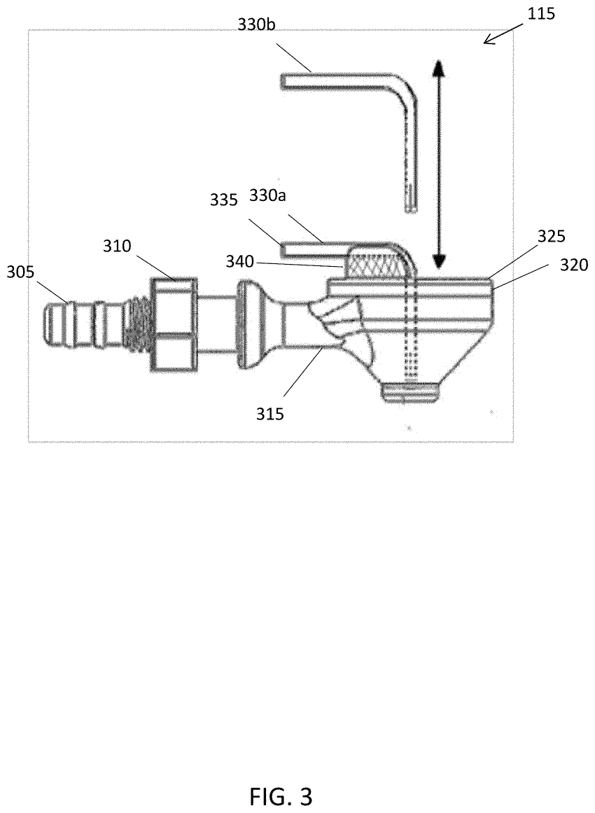

FIG. 1 depicts the micro-aerosol generator according to an embodiment of the present invention. The MAG 100 includes a vessel body 105 that supports one or more particle adjustment traps 110a and 110b as part of a bowl assembly. As will be explained in more detail with respect to FIG. 2, the number of PATs 110 can be configured in order to change particle size. More PATs 110 on the vessel body 105 yield smaller particle size within the dispersed micro-aerosol. The vessel body 105 includes a runout 120 for providing compressed air to one or more nozzle assemblies 115. The nozzle assemblies 115 are discussed in further detail with respect to FIG. 3. The runout 120 is in communication with an air compressor 140 for providing compressed air to the runout 120. The nozzle assemblies also are in communication with a fluid source 130 communicated via a liquid feed line 125. The fluid source 130 contains a decontamination fluid, such as HOCL. A drainage hose 135 returns non-dispersed decontamination fluid to the fluid source.

Using one or more nozzle assemblies 115, the nozzle assemblies 115 act as atomizers having an internal mixing chamber into which the decontamination fluid is fed and tangentially relative to walls of the internal mixing chamber compressed air is delivered. The outflow of the nozzle assemblies directed chordwisely relative to walls of the vessel body 105, with the projection of the central axis of the aerosol flame onto the vessel body 105 walls not intersecting with the upper edge of the walls at least during one turn. This results in reduction in aerosol particle size and assists in producing the micro-aerosol in conjunction with one or more PATs 110.

In operation, the decontamination fluid, such as HOCL, is introduced into the fluid source 130. Operation of the MAG 100 depends upon the size of the environment to be decontaminated and the power of the air compressor 140. For an air compressor 140 that produces 34 psi at 14.1 cubic feet per minute, the MAG 100 generates 15 ml of micro-aerosolized fluid into the air. For heavy coverage, there should be 0.1 ml per cubic foot of the environment into the air per minute. So, for example, for a 1,500 cubic foot space, the MAG 100 will operate for about ten minutes. After the run time, HVAC systems should not be running, allowing the micro-aerosol to be present undisturbed in the environment for about at least 30 minutes. For an air compressor 140 that operates at 17 psi at eight cubic feet per minute, the MAG 100 generates 5 ml of fluid in the air per minute, so run time is three times longer than with the 34 psi air compressor.

FIG. 2 depicts a plurality of particle adjustment traps 110 that are a part of the bowl assembly of the micro-aerosol generator 100 according to an embodiment of the present invention. An assembly of PATs 110 is made up of multiple curved rings that are stacked upon each other to adjust the amount and size of the micro-aerosol particles released into the environment being treated. The addition of each new PAT 110 will result in the incremental capture of larger particles than with fewer PATs 110. Furthermore, with the capture of increasingly larger particles the total micro-aerosol volume will decrease increasing a higher concentration of smaller particles to infiltrate the environment being treated. Absent a PAT 110, the MAG 110 will release particles between 0.1 micron and 40 microns. Each additional PAT 110 will decrease the size of the particles released into the space by approximately 7.5 microns. As an example, including two PATs 110 onto the MAG 100 will effectively reduce the largest size particle released into the air by 15 microns. As a result, the largest size particle released into the space will be no larger than 25 microns with the addition of the 2 PATs 110. The PAT 110 can effectively produce the above results up to the addition of 6 sections before de-rating the 7.5-micron increments.

FIG. 3 depicts a nozzle assembly 115 with a variable adjustment tip according to an embodiment of the present invention. The nozzle assembly includes a compressed air inlet 305, a nozzle base 315, nozzle top cover 325, nozzle o-ring 320, nut 310, and a variable adjustment tip 330 having a decontamination fluid inlet 335. In an exemplary embodiment consistent with the present invention, the variable adjustment tip 330 is made of stainless steel. Those of ordinary skill in the art after reading this description will appreciate that other materials can be used. The VAT 330 is supported by an adjustable stand-off 340 molded onto the nozzle top cover 325. The adjustable stand-off 340 keeps the VAT's 330 outlet tip at a predefined distance from the nozzle base's 315 outlet effectively controlling the range of particle sizes emitted by the nozzle assembly 115.

Height adjustments to the adjustable stand-off 340 vary the VAT 330 distance from the nozzles base outlet resulting in particle size change released by the nozzle assembly 115. Adjusting the VAT 330 inward or outward by 0.1 mm changes the particle sizes released by 5 microns. Inward adjustments decrease the particle size while outward adjustments increase the overall particle size. At the VAT's 330 nominal distance, VAT 330 distance without increase or decrease in size of the stand-off 340 is approximately 0.1 to 100 microns. As an example, decreasing the VAT 330 by 0.5 mm decreases the particle size released into the bowl by 25 microns resulting in the range released by the nozzle to be 0.1 to 75 microns.

The descriptions of the various embodiments of the present invention have been presented for purposes of illustration but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments described herein. Depending upon the pathogen load of the room, run time may be extended or shortened.

What is claimed is:

1. A method for providing a reduced pathogen environment, comprising:
providing a decontamination fluid; and
micro-aerosolizing the decontamination fluid within the environment for a predetermined period of time based on a volume of air in the environment, wherein the micro-aerosolized decontamination fluid has a particle size of between about 0.2 and about 1 micron.

2. The method of claim 1, wherein the decontamination fluid comprises hypochlorous acid.

3. The method of claim 1, wherein a plurality of the micro-aerosolized decontamination fluid has a particle size of about 1 micron.

4. The method of claim 1, further comprising adjusting the particle size of the micro-aerosolized decontamination fluid by adjusting a number of particle adjustment traps placed on a micro-aerosol generator used to micro-aerosolize the decontamination fluid.

5. The method of claim 1, further comprising adjusting the particle size of the micro-aerosolized decontamination fluid by adjusting a height of a variable adjustment tip within a nozzle assembly within a micro-aerosol generator used to micro-aerosolize the decontamination fluid.

6. The method of claim 1, further comprising adjusting the particle size of the micro-aerosolized decontamination fluid by:
adjusting a number of particle adjustment traps placed on a micro-aerosol generator (“MAG”) used to micro-aerosolize the decontamination fluid; and
adjusting a height of a variable adjustment tip within a nozzle assembly within a micro-aerosol generator (“MAG”) used to micro-aerosolize the decontamination fluid.

7. The method of claim 1, wherein particles within the micro-aerosolized decontamination fluid follow Graham's Law in flowing from areas of the environment having high concentration of particles to areas of the environment having low concentration of particles.

8. A micro-aerosol generator (“MAG”) comprising:
a vessel body;
a nozzle assembly mounted within the vessel body for micro-aerosolizing a decontamination fluid using compressed air; and
a first particle adjustment trap on the vessel body for reducing a size of particles micro-aerosolized by the nozzle assembly and emitted by the MAG.

9. The MAG of claim 8, wherein the first particle adjustment trap reduces the size of the particles by about 7.5 microns.

10. The MAG of claim 9, comprising a second particle adjustment trap on the first particle adjustment trap that reduces the size of the particles by an additional about 7.5 microns.

11. The MAG of claim 8, further comprising a variable adjustment tip for emitting the decontamination fluid within the nozzle assembly for micro-aerosolizing the decontamination fluid.

12. The MAG of claim 11, wherein the first particle adjustment trap reduces the size of the particles by about 7.5 microns.

13. The MAG of claim 12, comprising a second particle adjustment trap on the first particle adjustment trap that reduces the size of the particles by an additional about 7.5 microns.

14. A micro-aerosol generator (“MAG”) comprising:
a vessel body;
a nozzle assembly mounted within the vessel body for micro-aerosolizing a decontamination fluid using compressed air, the nozzle assembly comprising:
a nozzle base;
a nozzle o-ring on the nozzle base;
a nozzle top cover on the nozzle o-ring;
a variable adjustment tip having a decontamination fluid inlet for receiving decontamination fluid and an outlet for releasing decontamination fluid; and
an adjustable stand-off for adjusting a height of the variable adjustment tip.

15. The MAG of claim 14, wherein the variable adjustment tip comprises stainless steel.

16. The MAG of claim 14, wherein adjusting the variable adjustment tip inward by about 0.1 mm decreases a particle size of the micro-aerosolized decontamination fluid by about 5 microns.

17. The MAG of claim 14, wherein adjusting the variable adjustment tip outward by about 0.1 mm increases a particle size of the micro-aerosolized decontamination fluid by about 5 microns.

18. The MAG of claim 14, wherein a particle size of the micro-aerosolized decontamination fluid may be adjusted between about 0.1 microns and about 100 microns.

19. The MAG of claim 14, further comprising a first particle adjustment trap on the vessel body for reducing a size of particles micro-aerosolized by the nozzle assembly and emitted by the MAG.

20. The MAG of claim 19, further comprising a second particle adjustment trap on the first particle adjustment trap for further reducing a size of particles micro-aerosolized by the nozzle assembly and emitted by the MAG.
